(19) United States
(12) Patent Application Publication
(10) Pub. No.: US 2023/0241337 A1
(43) Pub. Date: Aug. 3, 2023

(54) SYSTEMS AND METHODS FOR OPTIMIZING SLEEP COMFORT

(71) Applicant: ResMed Pty Ltd, Bella Vista, NSW (AU)

(72) Inventors: Faizan Javed, Sydney (AU); Ryan Michael Kirkpatrick, Sydney (AU); Kenneth John Taylor, Sydney (AU); Gregory Robert Peake, Sydney (AU); Liam Holley, Sydney (AU); Nathan Zersee Liu, Sydney (AU)

(21) Appl. No.: 18/011,067

(22) PCT Filed: Sep. 16, 2021
(86) PCT No.: PCT/IB2021/058463

§ 371 Date: Dec. 16, 2022

Related U.S. Application Data
(60) Provisional application No. 63/080,682, filed on Sep. 19, 2020.

(51) Int. Cl. A61M 16/00

(52) U.S. Cl. CPC A61M 16/024; A61M 2016/0027

(57) ABSTRACT

A method for optimizing sleep for a user of a respiratory therapy system is described herein. The method includes receiving therapy instructions to be implemented using the respiratory therapy system for a sleep session. The therapy instructions include a plurality of prescribed control parameters, each of the plurality of prescribed control parameters having a value or a range of values. The method further includes receiving a desired sleep comfort level for the sleep session, and adjusting one or more of the values or the range of values of the plurality of prescribed control parameters, to one or more adjusted values or range of values based on the desired sleep comfort level. The adjustments to the adjusted values are implemented to aid the user in achieving the desired sleep comfort level.

### CROSS-REFERENCE TO RELATED APPLICATIONS

This application claims the benefit of, and priority to, U.S. Provisional Patent Application No. 63/080,682 filed on Sep. 19, 2020, which is hereby incorporated by reference herein in its entirety.

### TECHNICAL FIELD

The present disclosure relates generally to respiratory therapy systems and more particularly to systems and methods for optimizing sleep comfort for a user of a respiratory therapy system.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), mixed apneas, and hypopneas, Respiratory Effort Related Arousal (RERA), and chest wall disorders. These disorders are often treated using a respiratory therapy system. Users of the respiratory therapy system may have diagnosed or undiagnosed lung condition such as chronic obstructive pulmonary disease (COPD), emphysema, fibrosis, pneumonia, obstructive lung disease (OLD), restrictive lung disease (RLD), fixer upper airway obstruction (FUAO), asthma, and the like.

### SUMMARY

According to some implementations of the present disclosure, a method for optimizing sleep for a user of a respiratory therapy system. The method includes receiving therapy instructions to be implemented using the respiratory therapy system for a sleep session. The therapy instructions include a plurality of prescribed control parameters, each of the plurality of prescribed control parameters having a value or a range of values. The method further includes receiving a desired sleep comfort level for the sleep session, and adjusting one or more of the values or the range of values of the plurality of prescribed control parameters to one or more adjusted values or range of values based on the desired sleep comfort level. The adjustments to the adjusted values are implemented to aid the user in achieving the desired sleep comfort level.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 is a process flow diagram of a method for optimizing sleep for a user of a respiratory therapy system, according to some implementations of the present disclosure.

FIG. 4A is a plot of an adjusted pressure and adjusted ramp pressure, according to some implementations of the present disclosure.

FIG. 4B is a plot of an adjusted pressure response, according to another implementation of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

In order to mitigate some of these sleep-related and/or respiratory disorders, a user can be prescribed usage of a respiratory device or system. For example, a continuous positive airway pressure (CPAP) machine can be used to increase air pressure in the throat of the respiratory device user and to prevent the airway from closing and/or narrowing during sleep. Therefore, a goal of the therapy is to reduce the AHI to normal levels and improve the sleep quality of the user.

While these respiratory devices or systems can improve the sleep quality of the user, the comfort of the user is sometimes compromised. For example, while attempting to sleep a user may feel discomfort due to air being forced into their airways. Other discomforts might be related to sounds of the system, sweating or stickiness of a face mask, or discomfort and claustrophobia of wearing a mask. After a sleep session, a user can experience belching, stomach bloating, stomach distension and agonizing gas pains due to aerophagia. Aerophagia occurs when air enters the esophagus and goes into the stomach instead of the air entering the airways and to the lungs as intended. Other discomforts after a sleep session can include dryness of the nose, throat, or eyes as a result of leaks causing high air flow or poor humidification of the therapeutic air.

The discomfort experienced by a person who is prescribed use of a respiratory therapy system is often more pronounced when the user is first adopting a sleep therapy using the system. If the initial user experience is bad due to poor sleep comfort, the user is more likely to abandon the therapy and sacrifice sleep quality in pursuit of better sleep comfort. Deferment of treatment can lead to deterioration of quality of life due to the user not achieving high quality sleep.

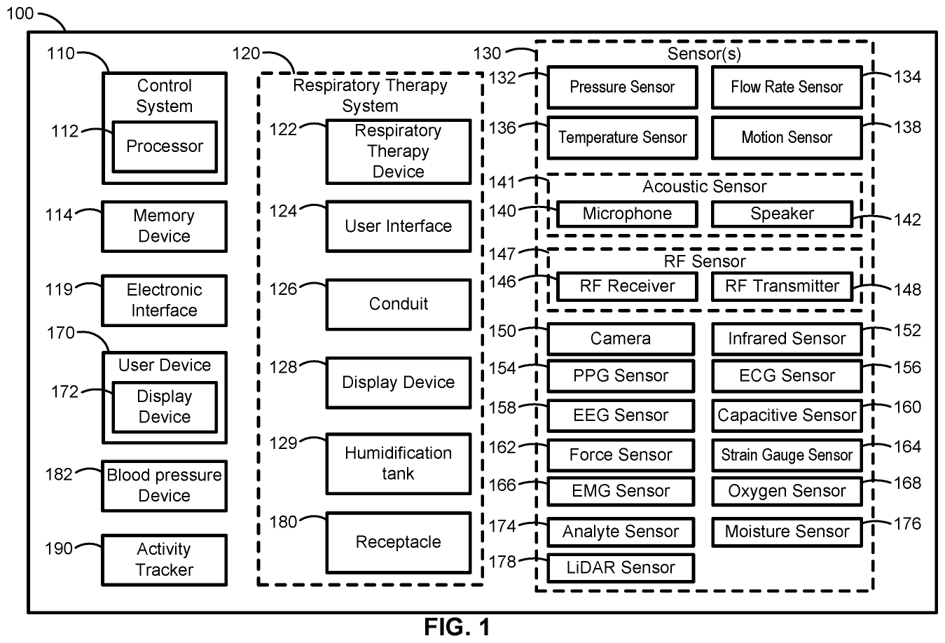

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120, a blood pressure device 182, an activity tracker 190, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with a user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy (RPT) device 122 (referred to herein as respiratory therapy device 122), a user interface 124, a conduit 126 (also referred to as a tube or an air circuit), a display device 128, a humidification tank 129, a receptacle 180 or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance of the user's airways at a controlled target pressure that is nominally positive with respect to atmosphere throughout the user's respiratory cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 has a blower motor (not shown) that is generally used to generate pressurized air that is delivered to the user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 4 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 liters/minute and about 150 liters/minute, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

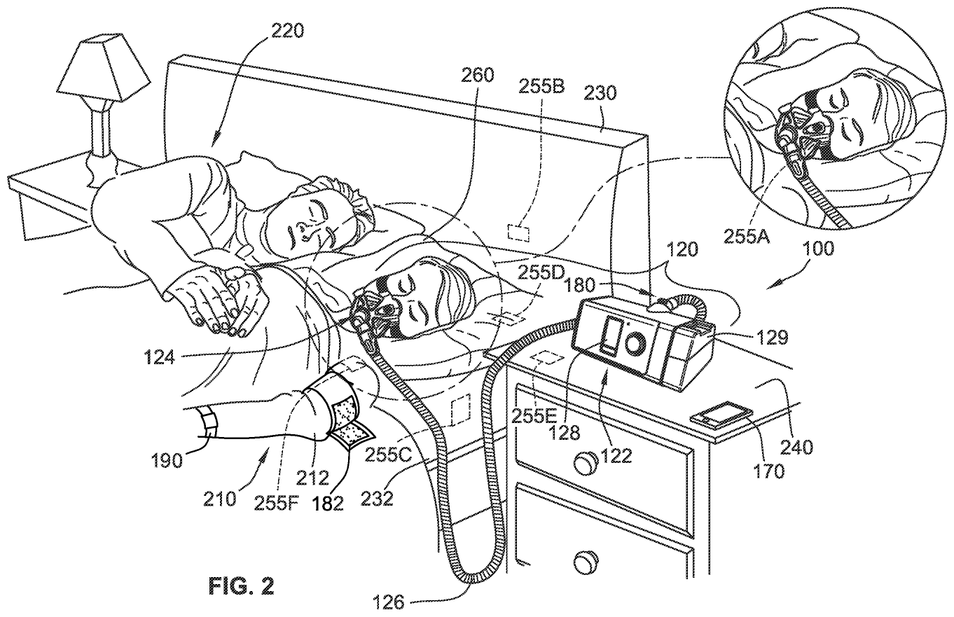

As shown in FIG. 2, in some implementations, the user interface 124 is a facial mask (e.g., a full facial mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps forming, for example, a headgear for aiding in positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 includes a mouthpiece (e.g., a night guard mouthpiece molded to conform to the teeth of the user 210, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit 126 for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory device 122, the temperature of the air being delivered by the respiratory therapy device 122, etc.) and/or other information (e.g., a sleep score and/or a therapy score, also referred to as a myAir™ score, such as described in US 2017/0311879 A1, which is hereby incorporated by reference herein in its entirety, the current date/time, personal information for the user 210, questions seeking feedback from the user and/or advice to the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include one or more vents (not shown) and a heater to heat the water in the humidification tank 129 in order to humidify the pressurized air provided to the user 210. Additionally, in some implementations, the conduit 126 can also include a heating element (e.g., coupled to and/or imbedded in the conduit 126) that heats the pressurized air delivered to the user 210. The humidification tank 129 can be fluidly coupled to a water vapor inlet of the air pathway and deliver water vapor into the air pathway via the water vapor inlet, or can be formed in-line with the air pathway as part of the air pathway itself. In some implementations, the humidification tank 129 may not include the reservoir of water and thus waterless.

In some implementations, the system 100 can be used to deliver at least a portion of a substance from the receptacle 180 to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 210. The APAP system automatically varies the pressurized air delivered to the user 210 based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Still referring to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a pressure signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, acoustic data, or both. Physiological data generated by one or more of the sensors 130 can be used by the control system 110 to determine a sleep-wake signal associated with the user 210 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, U.S. Pat. No. 10,492,720 B2, US 2014/0088373 A1, WO 2017/132726, WO 2019/122413, and US 2020/0383580 A1, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in U.S. Pat. No. 10,328,219 B2, which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user 210). The audio data from the microphone 140 can also be used to identify (e.g., using the control system 110) one or more sleep-related parameters or events experienced by the user 210 during the sleep session, as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user 210. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141, (e,g, a sonar sensor) as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230, and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or a combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the mouth of the user 210. For example, when the user interface 124 is a facial mask that covers the nose and mouth of the user 210, the analyte sensor 174 can be positioned within the facial mask to monitor the mouth breathing of the user 210. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example the air inside the bedroom of the user 210.

One or more Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having the LiDAR sensor 178 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 190, or any combination thereof. For example, the microphone 140 and the speaker 142 can be integrated in and/or coupled to the user device 170, and the pressure sensor 130 and/or the flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating cardiovascular data for determining one or more blood pressure measurements associated with the user 210. The blood pressure device 182 can include at least one of the one or more sensors 130 to measure, for example, a systolic blood pressure component and/or a diastolic blood pressure component.

In some implementations, the blood pressure device 182 is a sphygmomanometer including an inflatable cuff that can be worn by a user and a pressure sensor (e.g., the pressure sensor 132 described herein). For example, as shown in the example of FIG. 2, the blood pressure device 182 can be worn on an upper arm of the user 210. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff. More generally, the blood pressure device 182 can be communicatively coupled with, and/or physically integrated in (e.g., within a housing), the control system 110, the memory 114, the respiratory therapy system 120, the user device 170, and/or the activity tracker 190.

The activity tracker 190 is generally used to aid in generating physiological data for determining an activity measurement associated with the user 210. The activity tracker 190 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 190 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 190 is coupled (e.g., electronically or physically) to the user device 170.

In some implementations, the activity tracker 190 is a wearable device that can be worn by the user 210, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user 210. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and optionally the user device 170. Thus, various systems can be formed using any portion or portions of the components shown and described herein and/or in combination with one or more other components.

Referring generally to FIG. 2, a portion of the system 100 (FIG. 1), according to some implementations, is illustrated. The user 210 of the respiratory therapy system 120 and a bed partner 220 are located in a bed 230 and are laying on a mattress 232. The user interface 124 (also referred to herein as a mask, e.g., a full facial mask) can be worn by the user 210 during a sleep session. The user interface 124 is fluidly coupled and/or connected to the respiratory device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

In some implementations, the control system 110, the memory 214, any of the one or more sensors 130, or a combination thereof can be located on and/or in any surface and/or structure that is generally adjacent to the bed 230 and/or the user 210. For example, in some implementations, at least one of the one or more sensors 130 can be located at a first position 255A on and/or in one or more components of the respiratory therapy system 120 adjacent to the bed 230 and/or the user 210. The one or more sensors 130 can be coupled to the respiratory therapy system 120, the user interface 124, the conduit 126, the display device 128, the humidification tank 129, or a combination thereof.

Alternatively or additionally, at least one of the one or more sensors 130 can be located at a second position 255B on and/or in the bed 230 (e.g., the one or more sensors 130 are coupled to and/or integrated in the bed 230). Further, alternatively or additionally, at least one of the one or more sensors 130 can be located at a third position 255C on and/or in the mattress 232 that is adjacent to the bed 230 and/or the user 210 (e.g., the one or more sensors 130 are coupled to and/or integrated in the mattress 232). Alternatively or additionally, at least one of the one or more sensors 130 can be located at a fourth position 255D on and/or in a pillow that is generally adjacent to the bed 230 and/or the user 210.

Alternatively or additionally, at least one of the one or more sensors 130 can be located at a fifth position 255E on and/or in the nightstand 240 that is generally adjacent to the bed 230 and/or the user 210. Alternatively or additionally, at least one of the one or more sensors 130 can be located at a sixth position 255F such that the at least one of the one or more sensors 130 are coupled to and/or positioned on the user 215 (e.g., the one or more sensors 130 are embedded in or coupled to fabric, clothing 212, and/or a smart device worn by the user 210). More generally, at least one of the one or more sensors 130 can be positioned at any suitable location relative to the user 210 such that the one or more sensors 130 can generate sensor data associated with the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, her airway can narrow or collapse during sleep, reducing oxygen intake, and forcing her to wake up and/or otherwise disrupt her sleep. The CPAP machine prevents the airway from narrowing or collapsing, thus minimizing the occurrences where she wakes up or is otherwise disturbed due to reduction in oxygen intake. While the respiratory device 122 strives to maintain a medically prescribed air pressure or pressures during sleep, the user can experience sleep discomfort due to the therapy.

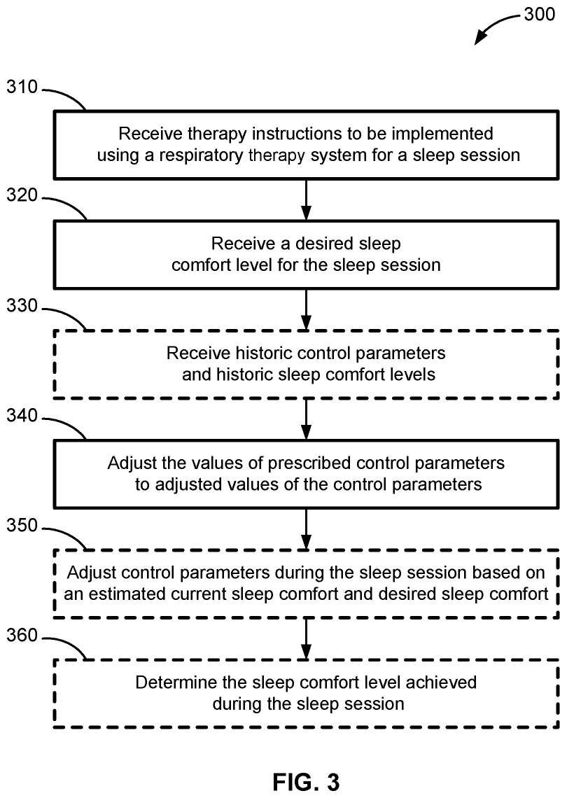

Referring to FIG. 3, a flow diagram illustrating a method 300 for optimizing sleep for a user of a respiratory therapy system according to some implementations of the description.

At step 310, therapy instructions to be implemented using the respiratory therapy system 120 for a sleep session are received. The therapy instructions can be entered or pre-programed into the respiratory therapy system 120 by a care provider or by the user 210. The therapy instructions can include a plurality of prescribed control parameters. For example, the therapy instructions can include a prescribed pressure and pressure ranges (e.g., a target pressure, a minimum and maximum pressure). The therapy instruction can also include a prescribed ramp rate, or ranges of ramp rates, to achieve the prescribed pressure or pressures.

In some implementations the control parameter includes a sound, an Expiratory Pressure Relief (EPR) setting, a humidification level, a device movement, a light turning on or changing in brightness, a fan turning on or changing in output, or any combination thereof.

Sounds that can be used as a control parameter include, without limitation, white noise, pink noise, brown noise, violet noise a soothing sound, music, an alarm, an alert, beeping, or a combination thereof. As used herein, some variants of the flat shaped white noise sound are referred to as pink noise, brown noise, violet noise, etc. In some implementations the sounds (e.g., white noise, pink noise, brown noise, violet, etc.) aid in masking noises from the respiratory therapy system or the environment. In some implementations, the sounds (e.g., soothing sounds and music) aid the user or bed partner to remain in a sleep state or can gently awaken or cause the user to change sleeping position, e.g., to a position to maintain a sleep session or sleep state.

In some implementations the sound can be provided by the one or more speakers 142 of system 100. Optionally, the system 100 includes multiple speakers 142 to provide localized sound emission. The speakers 142 can include in the ear speakers, over the ear speakers, adjacent to the ear speakers, ear buds, ear pods, or any combination thereof. The speakers 142 can be wired or wireless speakers (e.g., headphones, bookshelf speakers, floor standing speakers, television speakers, in-wall speakers, in-ceiling speakers, etc.). In some implementations, the speakers 142 are worn by the user 210 and/or the bed partner 220. In some such implementations, the provided speakers 142 can supply a masking noise without impacting the bed partner as the sound would be localized via the type of the speakers 142. In such implementations, respective localized speakers 142 could be provided for the respiration user 210 and/or the bed partner 220.

Optionally, the speaker 142 is attached to one or more of a strap or strap segments of the user interface 124. Thus, the user 210 (FIG. 2) and/or the bed partner 220 has the choice to perceive a relatively flat shaped white noise sound, or for a quieter (lower level and/or low pass filtered) shaped noise signal. In some such implementations, the higher frequency sounds/noises (e.g., “harsher” sounds) are reduced, while still providing masking sounds to the environmental noise. The system 100 can select an optimized set of fill-in sound frequencies to achieve a target noise profile. For example, if certain components of sound already exist in the frequency spectrum (e.g., related to a box fan in the room, a CPAP blower motor, etc.), then the system 100 can select fill-in sounds with sound parameters/characteristics that fill in the quieter frequency bands, for example, up to a target amplitude level. Thus, the system 100 is able to adaptively attenuate the higher and/or lower frequency components using active adaptive masking and/or as adaptive noise canceling such that the perceived sound is more pleasant and relaxing to the ear (the latter being more suited to more slowly varying and predictable sounds).

In general, EPR is a feature on some respiratory devices (e.g., CPAP machines) that allows users to adjust between different comfort settings to alleviate feelings of breathlessness some users experience. For example, a drop of 2 cm H₂O between inspiration and expiration. Where this feature can be manual, the EPR setting can be implemented by the system 100 without direct manual user input according to some implementations of this description to provide a desired sleep comfort.

In some implementations a control parameter is the humidity of the air delivered to the user 210. For example, the humidity level can be chosen to mitigate discomfort due to dryness of the sinuses or mouth. The humidity level can also be chosen to optimize a seal between the interface 124 and user 210.

A device that is actuated or moved as a control parameter can include, without limitation, a smart pillow, an adjustable bed frame, an adjustable mattress, a fan, an adjustable blanket, or any combination thereof. For example, the device is under control of controller 110. In some implementations the smart pillow, smart mattress, or adjustable blanket can include one or more inflatable compartments or bladders that can inflate or deflate. The actuated device can thereby change the orientation of user. For example, a pillow 260 can be a smart pillow including one or more inflatable bladders that change the orientation of user 210 if the user's head is in an orientation that increases the likelihood of increasing the AHI. In another or additional implementation, an adjustable bed frame can include sections that can rise or lower as driven by a motor and cause the user 210 to change orientation, e.g., forcing user 210 to roll from their side to their back. In some implementations, the fan, for example a fan placed on nightstand 240, a fan in a window or a ceiling fan, turns on responsive to the likelihood of an AHI increasing due to orientation of the user 210. In some implementations, the fan generates white noise. The fan can increase in speed and movement of air gradually, so as to not wake and/or disturb the user 210 and/or the bed partner 220 with a sudden change of air movement or sound from the fan.

In some implementations the control parameter is injection of a substance into the pressurized air to be being delivered to the user interface 124. For example, receptacle 180 can be charged with a substance, the receptacle having an outlet that is in direct or indirect fluid communication with the conduit 126. The substance can be configured or selected to invoke a physical reaction by the user 210. For example, the user 210 may change orientation.

Optionally, the substance can include a medicament, such as anti-inflammatory medicine, medicine to treat an asthma attack, medicine to treat a heart attack, etc. Generally, any type of medicament that is used to treat any ailment, symptom, disease, etc. can be delivered to the airway of the user 210. When the substance is a medicament, the substance generally includes one or more active ingredients, and one or more excipients. The excipients serve as the medium for conveying the active ingredient, and can include substances such as bulking agents, fillers, diluents, antiadherents, binders, coatings, colors, disintegrants, flavors, glidants, lubricants, preservatives, sorbents, sweeteners, vehicles, or any combinations thereof. The active ingredient is generally the portion of the medicament that actually causes the effect brought on by the medicament.

The substance can also optionally be an aroma compound (e.g., a substance that delivers scents and/or aromas to the airway of the user 210), a sleep-aid (e.g., a substance that aids the user 210 in falling asleep), a consciousness-arousing compound (e.g., a substance that aids the user 210 in waking up, also referred to as a sleep inhibitor), a cannabidiol oil, an essential oil (such as lavender, valerian, clary sage, sweet marjoram, roman chamomile, bergamot, etc.). The substance can generally be a solid, a liquid, a gas, or any combination thereof. The substance can alternatively or additionally include one or more nanoparticles.

In some implementations, the prescribed control parameters for a therapy is a function of the sleep stage or sleep architecture. For example, according to the user's prescription, in transitioning from an awake state to an N1 stage, a pressure ramp may be initiated to achieve a first target pressure from ambient pressure. The ramp can be implemented so as to gradually accustom the user 210 to the pressure change. During a following N2 and N3 the first target pressure may be maintained according to the user's prescription. Since during REM most users are more likely to experience an increase in apneas, a higher pressure from a previous sleep state is often prescribed for REM sleep stages. Accordingly, a ramp to the higher pressure can be implemented for REM sleep. The sleep state can be determined, as previously described, by monitoring physiological parameters using sensors (e.g., one or more sensors 130, blood pressure device 182, or activity tracker 190 of FIG. 1).

At step 320, a desired sleep comfort level is entered into the respiratory system 120. The desired sleep comfort may be selected based on how important the sleep comfort is to the user 210 for the sleep session. For example, a first time user 210 might be encouraged to select a high comfort level. A more experienced user 210 may not prioritize sleep comfort or they do not feel any significant discomfort when using the respiratory therapy system 120 with the prescribed control settings. In some instances, a user 210 may generally desire a high sleep comfort but has need for high quality sleep and is willing to have a lower comfort level for a specific sleep session and so selects a low desired sleep comfort level.

Alternatively or in addition to the user setting the desired sleep comfort level, in some implementations, the system 100 can automatically set a sleep comfort level for the user 210. The automatically set sleep comfort level can be based at least in part on data associated with the user 210 and or data based on the user's experience and/or length of time and/or hours using the respiratory therapy system 120 and/or sleep therapy. For example, the automatically set sleep comfort level can be set based on the number of days the user has been using sleep therapy, or the number of logged hours using a respiratory therapy system 120.

In some implementation, one aspect of sleep comfort relates to how the user 210 or a population of users rate a sleeping experience. The user or users can, after a sleeping session, rate the sleep comfort experience. The rating system can include different criteria including, for example, data of reported symptoms of aerophagia, difficulty in getting to sleep, dry sinuses/mouth, muscle soreness, and dry skin. The criteria can be subdivided and quantified such as by rating any pain due to aerophagia from low (e.g., mild gas), medium lingering discomfort (e.g., bloating), to high (e.g., cramps). Difficulty in getting to sleep can be rated as how many times the user might recall checking the time or noting noise or air pressure from the respiratory therapy system 120. Incidences of dry skin, sinuses or mouth, and muscle soreness can also be reported and used to rate the sleep comfort. The rating can be facilitated by a questionnaire with or without care provider's help. In some implementations, the questionnaire may also gather information such as whether the user 210 experiences restless sleep, insomnia, arousals during pressure therapy, bed partner's sleep comfort ratings (e.g., how it affects the bed partner) and other motivational feedback questions, such as increased activity in the following day after pressure therapy, etc. The questionnaire can be presented and the ratings entered by an interactive app via the user interface 124, a touchscreen of the respiratory device 122, a voice input, or an external device 170 (e.g., a smart phone).

An example of a short questionnaire for a user (Joe) rated sleep comfort is shown in Table 1.

TABLE 1
User Reported Sleep Comfort Data

| | |
|---|---|
| | Sleep Comfort |
| | Low-------------------------------High |
| User: Joe | Number rating |

| Date: Sep. 1, 2020 | 1 | 2 | 3 | 4 | 5 |
|---|---|---|---|---|---|
| Aerophagia | | x | | | |
| Dry sinuses/mouth | | | x | | |
| Dry skin (e.g., eye) | | | x | | |
| Going to sleep | x | | | | |
| Muscle soreness | | x | | | |
| Sleep quality | | | | x | |

The example questionnaire rates the sleep comfort (or discomfort) due to aerophagia, dry sinuses/mouth, dry skin, while going to sleep, muscle soreness, and sleep quality. The questionnaire rates these factors from low to high with 5 possible increments. Sleep quality relates to how well rested user 210 feels. For example, being clear headed and alert. Where the sleep quality does not characterize sleep comfort, it can often inversely relate to the sleep comfort. Including sleep quality in the sleep comfort rating can help in determining how much the sleep comfort can be modified without reducing the sleep comfort to levels where the therapy is not effective.

A total rated sleep comfort score can be determined as a function of the sleep comfort for each item listed in Table 1. Equation 1 shows one implementation of how a sleep comfort rating questionnaire can be used to provide a total rated sleep score.

Total Rated Sleep Comfort Score=(m₁)(aerophagia)+(m₂)(dry sinuses/mouth)+(m₃)(dry skin)+(m₄)(going to sleep)+(m₅)(muscle soreness)−(m₆)(sleep quality); Equation 1:

where m₁ is a weighting factor selected for aerophagia, m₂ is a weight factor selected for dry sinuses/mouth, m₃ is a weight factor for dry skin, m₄ is a weight factor for going to sleep, m₅ is the weight factor for muscle soreness, and m₆ is a weight factor for sleep quality. The Total Rated Sleep Comfort Score value can also be normalized e.g., to have a minimum of 0 or 1, and maximum of 5, 10, 20, 50 or 100. The weight factor relates to the importance of a specific item to sleep comfort. For example, the “aerophagia” item may ultimately be more important to sleep quality than the “going to sleep” item and a greater weight factor is selected or assigned for aerophagia than for going to sleep. The weight factors can be assigned by a first machine learning algorithm, for example, by providing data from multiple sleep sessions for one or more users. A “true” sleep comfort level for each individual sleep session is also used for training the first algorithm. Here the “true” sleep comfort refers to a user, or users, provided overall assessment of the sleep comfort, which can be assigned a value for training the first machine learning algorithm.

The first machine learning algorithm can also determine the overall function, including items other than those listed in Table 1, to provide the most accurate total rated sleep comfort score. In some implementation, the first machine learning algorithm can learn how an individual user 210 rates criteria that is subjective. For example, a first user may rate a sore muscle as more detrimental to sleep comfort than a second user. The first algorithm can learn this difference between the first and second user and accordingly modify the function to determine the Total Rated Sleep Comfort Score, depending on which user is using the respiratory therapy system 120. For example, if equation 1 is used, the weighing factor m₅ associated with muscle soreness for the first user would be less than the same weighing factor for the second user.

In some implementations, an aspect of sleep comfort relates to monitoring a user 210 or users of the respiratory therapy system 120 with sensors, such as sensors 130, during sleep sessions. In some implementations, the sleep architecture for the sleep session is determined as previously described, and the achieved sleep comfort level is determined as a function of sleep architecture. For example, a user 210 may be detected as moving during a non-REM sleep session. A user may be unconsciously trying to, or succeeding at, remove a user interface 124 in an N1 or other sleep phase. In some implementations, a user may be in a sleep position in a non-REM sleep phase (e.g., back vs side) which leads to poor sleep comfort. These activities can be factors influencing sleep comfort and can be monitored using sensors such as motion sensor 138, camera 150, or microphone 140, blood pressure device 182, activity tracker 190, or any combination thereof. Other indicators of sleep comfort that can be monitored can include noises from the respiratory therapy system 120 such as a pump or a leak at the user interface 124 (e.g., a mask leak). The ambient temperature as measured by temperature sensor 136 can also be indicative of sleep comfort. For example, a temperature that is higher or lower than an ideal temperature (e.g., 63° F.) can impact sleep comfort. While a user 210 may not instantly know how the various sensor monitored factors are impacting their sleep, the sensors can monitor these in real time as well as track and provide the data for analyses after the sleep session.

An example of some factors that can be monitored using sensors is shown by Table 2.

TABLE 2
Measured Sleep Comfort factors.

| User: Joe | |
|---|---|
| Date: Sep. 1, 2020 | Value |
| # non-REM | 50 |
| movements | |
| Percent time on | 40% |
| back vs side | |
| Average | 65° F. |
| temperature of | |
| room | |
| # of mask leaks | 5 |
| # incidences of | 2 |
| noises >40 dB | |
| AHI | 10 |

Table 2 lists the number of non-REM movements, percent time on back vs side, average room temperature, number of mask leaks, number of incidence of noises above a whisper (e.g., about 40 dB), and AHI. Where AHI is not a direct measure of sleep comfort it can be inversely proportional or otherwise counter to the sleep comfort. Including AHI can provide a balancing consideration since reducing AHI is an important objective of sleep therapies for various sleeping disorders.

A total measured sleep comfort score can be determined as a function of these sensor measurable factors. Any useful function can be implemented. An embodiment of a simple function is shown by equation 2.

Total Measured Sleep Comfort Score=(m₇)(N-REM Movements)+(m₈)(% Time on Back/Side)+(m₉)(Ave. Rm. Temp)+(m₁₀)(#of Mask Leaks)+(m₁₁)(#Noises)−(m₁₂)(AHI); Equation 2:

where m₇ is a weighting factor selected for N-REM movements, m₈ is a weight factor selected for % time on back/side, m₉ is a weight factor for average room temperature, m₁₀ is a weight factor for number of mask leaks, mu is a weight factor for number of noise incidences >40 dB, and m₁₂ is a weight factor for AHI. The weight factors, and the overall form of the function, can be determined by using a second machine learning algorithm. The Total Measured Sleep Comfort Score value can also be normalized e.g., to have a minimum of 0 or 1, and maximum of 5, 10, 20, 50 or 100. Data from the user or multiple users over multiple sleep sessions can be input into the second machine learning algorithm. A true sleep comfort can be used for training the second machine learning algorithm. In some implementations, the first machine learning algorithm provides a rated sleep comfort which can be used to train the second machine learning algorithm to determine the measured sleep comfort. For example, the rated sleep comfort from the first algorithm is used as the true sleep comfort for training the second algorithm.

In some implementations data from user 210 rated sleep comfort (e.g., Table 1) is combined with data from sensor measured sleep comfort factors (e.g., Table 2). For example, an overall sleep comfort score can be determined using user reported sleep comfort and measured sleep comfort. For example, a function for an Overall Sleep Comfort Score can be a combination of equation 1 and 2. The Overall Sleep Comfort Score value can also be normalized e.g., to have a minimum of 0 or 1, and maximum of 5, 10, 20, 50 or 100. In some implementations the first algorithm and second algorithm are combined as a single machine learning algorithm.

Returning to FIG. 3 and step 320, the desired sleep comfort level can be a value selected from a series of incrementally increasing values between a first value, indicative that the comfort experience is not important to the user, and a second value, indicative that the user desires the best possible sleep comfort experience. The values can be scaled similarly to the sleep comfort score that is used i.e., the Rated Sleep Comfort Score (e.g., Table 1, equation 1), the Measured Sleep Comfort Score (e.g., Table 2, equation 2) or the Overall Sleep Comfort Score (e.g., the combination of the Rated Sleep Comfort Score and the Measured Sleep Comfort Score). For example, the minimum and maximum values for the various scores correspond to the minimum and maximum values the user 210 can select for the desired sleep comfort. The values can be on a continuous scale, such as an analog volume control, or they can be digital. In other implementations, the sleep comfort can have digital value. For example, the values can be integers between 1 and 10, where 1 is indicative that the user does not desire an improvement in sleep comfort, and where 10 indicates the user desires the best possible sleep comfort experience. The sleep comfort level can be selected or dialed in according to the desired sleep comfort of the user.

While in some implementations the desired comfort level is selected when a user 210 goes to bed for a sleep session, in some other implementation the comfort level can be changed by the user 210 during the sleep session. For example, a user 210 may wake up after some combination of non-REM and REM sleep and decide that they are uncomfortable or can't get back to sleep. The user 210 can accordingly decide to increase the sleep comfort level. Alternatively, the user 210 may wake up during a sleep session and notice the time is 4 am and decide they need to get a couple more hours of high quality sleep so they decrease the sleep comfort to improve their sleep quality.

As shown by step 330, in some optional implementations, historic control parameters and historic sleep comfort levels can be entered into the respiratory therapy system 120. As used here, “historic” relates to one or more previous sleep sessions. For example, a historic sleep comfort might be a user selected value of 8 (e.g., on a scale of 1-10) which the user may have selected for a sleep session just prior to the current sleep session. In this instance, the historic control parameters are control parameters from the previous sleep session implemented using the respiratory therapy system 120 to target the desired sleep comfort of 8. A user may, after assessing the previous sleep session, determine that the comfort level actually achieved (the historic sleep comfort) is lower or higher than what they had entered. The received historic parameters and sleep comfort can be used for the current sleep session to more accurately achieve the desired sleep comfort for the sleep session where the user input indicates a gap between. For example, the user can select a higher or lower sleep comfort level based on their personal experience to self-titrate the desired sleep comfort.

In some implementations, the use of historic data relates to training of the system and can be implemented with the use of artificial intelligence. For example, a third machine learning algorithm can include the data used in the first machine learning algorithm (user reported sleep comfort), the second machine training algorithm (user measured sleep comfort), and a historic adjusted control parameter. In some implementations, the third machine learning algorithm is a combination of or includes elements from the first and second machine training algorithms.

In step 340, the control parameters are adjusted from the prescribed control parameters and the adjusted control parameters are implemented during the sleep session. Where the prescribed control parameters are implemented to improve the sleep quality of the user 210, the adjusted control parameters are implemented to achieve the desired sleep comfort level of the user 210.

In some implementations, the received therapy instructions are provided to aid a user in achieving a target therapy parameter during a sleep session, and the adjusted one of more values or range of values of the plurality of control parameters provides a therapy parameter that is different from the target therapy parameter. In some implementations, the adjusted one or more of the values or the range of values of the plurality of control parameters provides a therapy parameter that is greater than the target therapy parameter. In some other implementations, the adjusted one or more of the values or the range of values of the plurality of control parameters provides a therapy parameter that is less than the target therapy parameter.

In some implementations the received therapy instructions are provided to aid a user in achieving a target AHI for the user during the sleep session. While in some implementations, the achieved AHI is about the same as the target for the therapy, in some other implementation the adjusted control parameter can lead to an AHI that is greater than (e.g., worse than) the target AHI.

The sleep comfort can therefore be improved at the expense of a degradation in the quality of the sleep. In some implementations, the control parameters are adjusted to maximized the sleep quality and maximize the sleep comfort. For example, maximizing sleep quality and sleep comfort can be a feature of a machine learning algorithm such as the third machine learning algorithm.

In some implementations the pressure, range of pressures, or pressure ramps are adjusted up or down from the prescribe pressure, ranges of pressures, or pressure ramps. In some implementations, the pressure, range of pressures, or pressure ramps are adjusted to be lower than the prescribed pressure for at least a portion of the sleep session. In some implementations, the pressure, range of pressures, or pressure ramps are adjusted to be higher than the prescribed pressure for at least a portion of the sleep session. In some implementations, the pressure or range of pressures are adjusted to be higher than the prescribed pressure for at least a portion of the sleep session, and the average adjusted pressure during the entire sleep session is lower than the average prescribed pressure for the entire sleep session.

In some implementations, sounds that are provided by the one or more speakers 142 of system 100 are adjusted from prescribed sounds. In some embodiments, white noise, pink noise, brown noise, violet noise a soothing sound, or music is increased in volume, decreased in volume, increased in duration or decreased in duration from the prescription. In some implementations an alarm or alert that indicates a poor sleeping position with respect to sleep quality is turned off. The silencing of the alarm allows the user 210 to continue sleeping, albeit in a poor position, but providing more sleep comfort.

In some implementations, an EPR setting is adjusted up or down from a prescribed value. For example, where an EPR setting is a drop of 0.5 cm H₂O between inspiration and expiration as prescribed, the adjusted setting can be 1 cm H₂O, 1.5 cm H₂O, or 2.0 cm H₂O.

In some implementations, the humidity of the air delivered to the user 210 is adjusted up or down from a prescribed value. For example, in some implementations the humidity is increased to mitigate discomfort due to dry skin or dry sinuses/mouth. In some other implementations the humidity is decreased to mitigate discomfort due to the user 210 feeling discomfort due to slickness or stickiness of the user interface 124 (e.g., a face mask). The decrease in humidity can lead to a decrease in sleep quality, for example, due to increase face leaks, but the sleep comfort is increased.

In some implementations an adjusted control parameter includes a device that is actuated or moved. In a prescribed therapy the device may cause a user 210 to change position to reduce or avoid a mask leak. Where a user 210 may tend to move to a position that causes a mask leak, the repeated actuation of the device to try and force the user to a different position might cause sleep discomfort. For, example, forcing the user to assume a position that causes muscle soreness or causes aerophagia.

In implementations where the control parameter is injection of a substance into the pressurized air to be being delivered to the user interface 124, the adjustment can be a decrease or increase in the delivered substance. For example, a consciousness-arousing compound can be prescribed to limit the sleep session. As adjusted, the delivery of the substance can be delayed to a later time in the sleep session thereby prolonging the sleep session and improving the sleep quality.

In some implementations the prescribed control parameters maintain an ideal sleep architecture. The adjusted control parameters can change the ideal sleep architecture. For example, with adjusted control parameters less REM might be occur due to an increase in apneas occurring.

In some implementations, the selected desired sleep comfort level does not provide any measurable increase in sleep quality but can be used to allow a user 210, such as a first time user, to adopt the sleep therapy. The user 210 can gradually, optionally with guidance from a care provider, decrease the comfort level to improve the sleep quality in a “weaning” process. In some implementations, the weaning process is part of a program extending over several days, weeks or months and can be an automatically implemented feature of the control system 110. In some implementations, the weaning program can be a feature of one or more of the machine learning algorithms described herein.

Step 350 shows an optional implementation wherein the control parameters are adjusted during the sleep session responsive to a current sleep comfort and the desired sleep comfort. The current sleep comfort is an estimated sleep comfort of the user and does not require any direct or conscious input from the user. For example, the current sleep comfort can be determined by monitoring the user 210 using sensors such as sensors 130, blood pressure device 182, or activity tracker 190 of FIG. 1. Where Table 2 shows measured sleep comfort for an entire sleep session, the various factors that can be monitored using sensors can be sampled and tallied during a sleep session. How these factors change during the sleep session can be used to predict the sleep comfort that will be achieved during the sleep session. Where the trajectory of the predicted sleep comfort based on the current sleep comfort deviates from the desired sleep comfort level, corrective action can be implemented. The corrective action can be implemented by modification of the control parameters. For example, if the temperature is high and predicted to decrease the sleep comfort, a thermostat can be reset or a fan turned on. If a muscle soreness is predicted due to a user 210 sleeping position, a device such as a smart pillow, smart mattress, or adjustable blanket can be activated to cause the user 210 to change position. The prediction can be implemented using a prediction algorithm. The prediction algorithm can be a fourth machine learning algorithm, that can include the previously described first, second and third algorithms.

Step 360 is an optional step that includes determining the sleep comfort level achieved by user 210 in the sleep session. The sleep comfort level achieved can be determined as previously described, for example, using the user rated sleep comfort and measured sleep comfort. Optionally, the sleep comfort is determined using a fifth machine learning algorithm that can be any combination of the first, second, third and fourth machine learning algorithms previously discussed. In some embodiments the sleep comfort is reported out to the user, for example through external device 170.

According to some implementations, any of the plurality of prescribed control parameters can be adjusted to improve the sleep comfort. For example, the plurality of control parameters can include a prescribed pressure, a range of prescribed pressures, a range of prescribed pressures ramps, and a range of prescribed step pressures changes, and one or more of the prescribed pressure, the range of prescribed pressures, the range of prescribed pressures ramps, and the range of prescribed step pressures changes are adjusted to improve the sleep comfort. In some implementations, the prescribed pressure is adjusted to an adjusted pressure that is less than the prescribed pressure or range of prescribed pressures. In some implementations, the prescribed pressure range is adjusted to a range of pressures that is lower than the prescribed range of pressures. For example, the average or mean of the prescribed pressure range can be adjusted to be lower, or one or more of the maximum or minimum pressure can be adjusted lower.

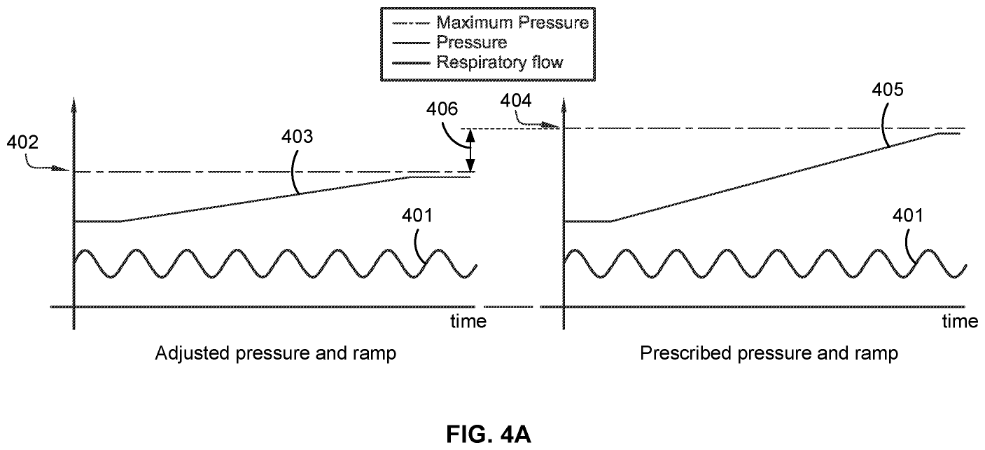

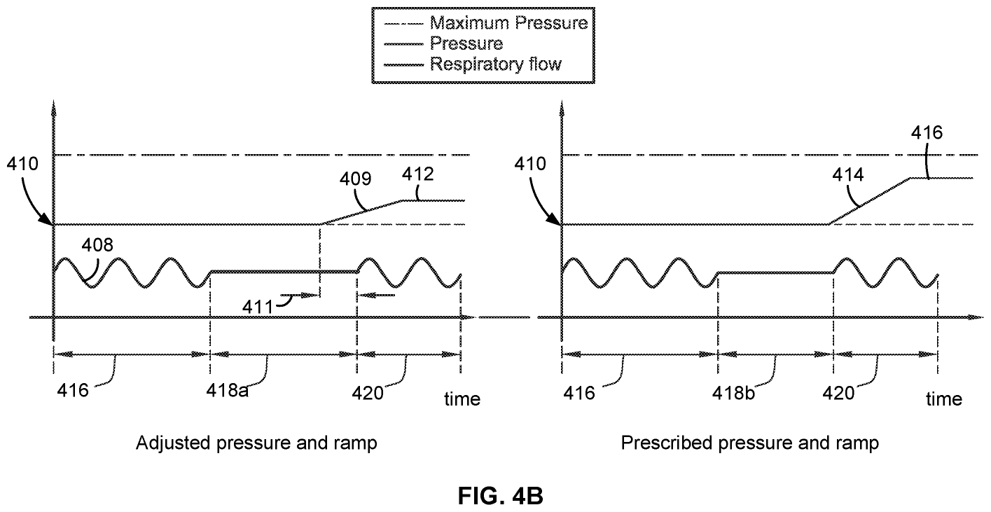

FIG. 4A are plots showing an implementation according to some aspects of the disclosure. The left side plot shows an adjusted target maximum pressure 402, an adjusted pressure ramp 403, and a respiratory flow 401. The right side plot shows the therapeutic prescribed maximum pressure 404, the prescribed pressure ramp 405, and the respiratory flow 401. A user may be prescribed a maximum pressure 404 to be implemented with respiratory therapy system 120. The target maximum pressure 404 can be, for example, 15 mm H₂O. This pressure is prescribed to provide a target AHI, such as 10 or less per sleep session. The prescribed target AHI and pressure can be determined, for example, during a titration experiment supervised by a care provider. The user 210 may find the prescribed pressure 404 and/or ramp 405 reduces their sleep comfort. For example, the user 210 may have symptoms of aerophagia after a sleep session where the maximum pressure 404 is implemented. Alternatively, or additionally, the user 210 may find ramp 405 increases the pressure too rapidly, making it difficult to fall asleep. The adjusted maximum pressure 402 and the adjusted pressure ramp 403 are responsive to the user 210 selecting a desired sleep comfort level. For example, a user feeling bloated when the prescribed pressure ramp 405 is implemented, selects a sleep comfort level to decrease the bloating and increase sleep comfort. Likewise, the more gradual increase in pressure ramp 403 as compared to prescribed pressure ramp 405 can provide a gentler transition for user 210 to go to sleep. While the adjusted target maximum pressure 402 and the adjusted pressure ramp 403 can provide better sleep comfort as compared to the prescribed target maximum pressure 404 and the prescribed pressure ramp 403, the sleep quality can be reduced. For example, the AHI achieved can be higher using prescribed maximum pressure 404 as compared to the AHI achieved using adjusted maximum pressure 402.

As another example, the user 210 may find that the target pressure ramp 405 starts at a pressure that is too low, which does not deliver a large enough quantity of breathing air and creates an uncomfortable feeling of hungering for air, called “air hunger”. This may also reduce sleep comfort and make it difficult to fall asleep. The target pressure ramp 405 may then be adjusted by the user to a desired sleep comfort level such that there is an appropriate adjusted pressure ramp 403, which enables the user 210 to provide sufficient breathing air and go to sleep, while on therapy.

The plots illustrated in FIG. 4A also show another aspect according to some implementations. A delta 406 between the prescribed target maximum pressure 404 and the adjusted target maximum pressure 402 is shown. Where a higher pressure causes more sleep discomfort, a larger delta 406 indicates the user 120 has selected a higher desired sleep comfort. In comparison, a smaller delta 406 would indicate a selected a lower sleep comfort. Although shown as applied to pressure, other control factors can be similarly manipulated and the delta between a prescribed and adjusted value is responsive to the desired sleep comfort level. In some implementation, an increase in a control parameter will provide better sleep comfort. For example, if the prescribed control parameter is a concentration of a medicament added via receptacle 180, an increase of the medicament, while possibly improving sleep comfort, could cause more apneas. In this instance, the increase in the medicament is an adjustment of a control parameter to a higher level from the prescribed control parameter.

FIG. 4B shows an implementation according to another aspect of the description. The left side plot shows an adjusted pressure and ramp profile. The left side plot shows a respiratory flow 408 and time segments 416, 418a and 420. After time segment 416, an apnea is shown in time segment 418a. In response to the apnea, a ramp 409 from an initial pressure 410 to a second higher pressure 412 is implemented by respiratory system 120. After a delay 411, the apnea is stopped and regular breathing continues in time segment 420. The right side plot shows a prescribed pressure and ramp profile. After time segment 416 at pressure 410, an apnea occurs in time segment 418b. In response a ramp 414 to a higher target pressure of 416 is implemented. Ramp 414 is steeper (positive) than ramp 409, and the pressure 416 is also higher than pressure 412. Because of the more aggressive control parameters used in the prescribed therapy, the apnea segment 418b is shorter than the apnea segment 418a. The segment 418b is shorter than 418a according to this implementation because the apnea is more quickly stopped after implementation of the pressure ramp 414, than after implementation of pressure ramp 409. Specifically, no delay 411, shown in the left side plot, is seen in the right side plot. Although the more aggressive control parameters implemented using the prescribed pressure ramp 414 and pressure 416 can more effectively eliminate an apnea, this can lead to sleep discomfort. The less aggressive pressure ramp 409 and pressure 412 reduces the discomfort.

One or more elements or aspects or steps, or any portion(s) thereof, from one or more of any of claims 1-36 below can be combined with one or more elements or aspects or steps, or any portion(s) thereof, from one or more of any of the other claims 1-36 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for optimizing sleep for a user of a respiratory therapy system, the method comprising:
receiving therapy instructions to be implemented using the respiratory therapy system for a sleep session, the therapy instructions including a plurality of prescribed control parameters, each of the plurality of prescribed control parameters having a value or a range of values for achieving a medically prescribed therapy;
receiving a desired sleep comfort level for the sleep session, wherein the desired sleep comfort level is indicative of a sleep comfort experience desired by the user for the sleep session; and
adjusting, based on the desired sleep comfort level, two or more of the values or the range of values of the plurality of prescribed control parameters to obtain two or more adjusted values or range of values, the two or more adjusted values or range of values departing from the medically prescribed therapy to aid the user in achieving the desired sleep comfort level.

2. The method of claim 1, wherein the received therapy instructions are provided to aid the user in achieving a target therapy parameter during the sleep session, and the adjusted two or more of the values or the range of values of the plurality of control parameters provides a therapy parameter that is different from the target therapy parameter.

3. The method of claim 2, wherein the adjusted two or more of the values or the range of values of the plurality of control parameters provides a therapy parameter that is greater than the target therapy parameter.

4. The method of claim 2, wherein the adjusted two or more of the values or the range of values of the plurality of control parameters provides a therapy parameter that is less than the target therapy parameter.

5. The method of claim 2, wherein the target therapy parameter includes an Apnea Hypopnea Index (AHI), oxygen saturation level (SpO₂), snoring, choking, heart rate, labored breathing, restlessness, or any combination thereof.

6. The method of claim 3, wherein the target therapy parameter is a target Apnea Hypopnea Index (AHI) for the user during the sleep session, and the adjusted two or more of the values or range of values of the plurality of control parameters causes the user to experience an actual AHI that is greater than the target AHI.

7. The method of claim 1, wherein the plurality of control parameters includes a prescribed pressure, a range of prescribed pressures, a prescribed pressure ramp rate, a range of prescribed pressures ramp rates, a range of prescribed step pressures changes, or any combination thereof.

8. The method of claim 7, wherein the adjusting the two or more values or the range of values of the plurality of prescribed control parameters includes one of: (i) adjusting the prescribed pressure to an adjusted pressure that is less than the prescribed pressure, (ii) adjusting the range of prescribed pressures to a range of pressures with a median pressure that that is lower than a median pressure of the range of prescribed pressures, or (iii) adjusting the prescribed pressure ramp rate to an adjusted pressure ramp rate that is less than the prescribed pressure ramp rate.

9-10. (canceled)

11. The method of claim 1, wherein the desired sleep comfort level is automatically set by respiratory therapy system for the user for the sleep session.

12. The method of claim 1, wherein the desired sleep comfort level is a value selected from a series of incrementally increasing values between a first value, indicative that the comfort experience is not important to the user, and a second value, indicative that the user desires the best possible sleep comfort experience.

13. (canceled)

14. The method of claim 12, wherein a difference, a delta, between at least one of the prescribed control parameters and the corresponding adjusted control parameters, is minimized by selection of the first value, and the delta is maximized by selection of the second value.

15. The method of claim 12, wherein the sleep comfort experience is determined by user reported sleep comfort data, one or more sensors, or a combination thereof.

16. The method of claim 15, wherein the user reported sleep comfort data includes data associated with: discomfort due to incidents of bloating after a sleep session, discomfort due to skin irritation ascribed to a mask leak after a sleep session, discomfort due to dry mouth or dry sinuses after a sleep session, discomfort due to restlessness or arousals during therapy, discomfort due to an initial therapy pressure causing air hunger, discomfort due to rapid ramp rate, insomnia or difficulty in getting to sleep during a sleep session, a caretaker or sleep partner observation of discomfort of the user after a sleep session, incidence of increased activity in the following day after therapy, or any combination thereof.

17. The method of claim 16, wherein the user reported sleep comfort data includes values selected from a series of incrementally increasing values between a first value, indicative that the sleep comfort data does not change the sleep comfort, and a second value, indicative that the sleep comfort data has a negative effect on the sleep comfort.

18. The method of claim 17, wherein the user reported sleep comfort data includes an integer between a first value of 1 and a second value of 10.

19. The method of claim 15, wherein the one or more sensors include a pressure sensor, a flow rate sensor, a temperature sensor, a motion sensor, an acoustic sensor, a camera, a PPG sensor, an EEG sensor, ECG sensor, a force sensor, an EMG sensor, an analyte sensor, an infrared sensor, a capacitive sensor, a strain gauge sensor, an oxygen sensor, and a moisture sensor, or any combination thereof; and wherein the one or more sensor are (i) positioned within or coupled to the respiratory therapy system, (ii) separate and distinct from the respiratory therapy system, or (iii) both (i) and (ii).

20. (canceled)

21. The method of claim 15, further comprising determining, for the sleep session, an achieved sleep comfort level using the sleep comfort experience for the sleep session.

22. The method of claim 21, further comprising determining a sleep architecture for the sleep session using physiological data associated with the user that is generated by the one or more sensors, and wherein the achieved sleep comfort level is determined as a function of the determined sleep architecture.

23. The method of claim 21, wherein the achieved sleep comfort level is a function of a sleep state, and one or more of motion, audible sounds, and a sleeping position, as detected by the one or more sensors.

24. The method of claim 1, further comprising adjusting one or more devices in an environment of the user to modify, ambient lighting, ambient noise, a soothing sound, the ambient temperature, air flow, the position of the user by a smart pillow or mattress, or a combination thereof, to aid the user in achieving the desired sleep comfort level.

25. The method of claim 1, further comprising using a machine learning algorithm to aid the user in achieving the desired sleep comfort level, the machine learning algorithm using historical adjusted values or a range of values and associated historical desired sleep comfort levels as inputs, the historical adjusted values or range of values and the historical desired sleep comfort levels provided from one or more prior sleep sessions.

26. The method of claim 25, wherein the historical adjusted values or range of values and the historical desired sleep comfort levels are sourced from a plurality of users of respective respiratory therapy systems.

27. The method of claim 1, wherein the receiving the desired sleep comfort level includes the user communicating the desired sleep comfort level to the respiratory therapy system or an external device communicatively connected to the respiratory therapy system.

28. The method of claim 15, further comprising:
receiving, from the one or more sensors, physiological data associated with the user;
determining a current sleep state of the user based on the received physiological data;
determining, based at least in part on the received physiological data, an estimated current sleep comfort of the user during the current sleep state of the user; and
during the current sleep state, adjusting at least one of the one or more values or the range of values of the plurality of prescribed control parameters, to an adjusted value or an adjusted range of values based at least in part on the estimated current sleep comfort of the user and the received desired sleep comfort level.

29. The method of claim 28, wherein the current sleep comfort of the user is a function of motion of the user during the sleep session, audible sounds detected during the sleep session, sleeping position of the user during the sleep session, of any combination thereof.

30. The method of claim 28, further comprising adjusting one or more external devices in an environment of the user to modify, ambient lighting, ambient noise, a soothing sound, the ambient temperature, air flow, the position of the user by a smart pillow or mattress, or a combination thereof.

31-36. (canceled)

37. A system for optimizing sleep for a user of a respiratory therapy system, the system comprising:
a memory having stored thereon machine readable instructions; and
a control system including one or more processors configured to execute the machine-readable instructions to:
receive therapy instructions to be implemented using the respiratory therapy system for a sleep session, the therapy instructions including a plurality of prescribed control parameters, each of the plurality of prescribed control parameters having a value or a range of values for achieving a medically prescribed therapy;
receive a desired sleep comfort level for the sleep session, wherein the desired sleep comfort level is indicative of a sleep comfort experience desired by the user for the sleep session; and
adjust, based on the desired sleep comfort level, two or more of the values or the range of values of the plurality of prescribed control parameters to obtain two or more adjusted values or range of values, the two or more adjusted values or range of values departing from the medically prescribed therapy to aid the user in achieving the desired sleep comfort level.

38. The system of claim 37, wherein the received therapy instructions are provided to aid the user in achieving a target therapy parameter during the sleep session, and the adjusted two or more of the values or the range of values of the plurality of control parameters provides a therapy parameter that is different from the target therapy parameter.

39. The system of claim 37, further comprising one or more sensors, and wherein executing the machine-readable instructions further configure the control system to:
receive, from the one or more sensors, physiological data associated with the user;
determine a current sleep state of the user based on the received physiological data;
determine, based at least in part on the received physiological data, an estimated current sleep comfort of the user during the current sleep state of the user; and
during the current sleep state, adjust at least one of the one or more values or the range of values of the plurality of prescribed control parameters, to an adjusted value or an adjusted range of values based at least in part on the estimated current sleep comfort of the user and the received desired sleep comfort level.
